(19) United States
(12) Patent Application Publication
(10) Pub. No.: US 2011/0015514 A1
(43) Pub. Date: Jan. 20, 2011

(54) MEDICAL IMAGING METHOD AND SYSTEM FOR PROVIDING A FINITE-ELEMENT MODEL

(75) Inventors: Wafa Skalli, Paris (FR); David Mitton, Kremlin Bicetre (FR); Jean Dubousset, Paris (FR); Francois Lavaste, Saint Michel Sur Orge (FR)

Correspondence Address: Osler, Hoskin & Harcourt LLP (Plasseraud), 1000 de la Gauchetiere Street West, Suite 2100, Montreal, QC, H3B 4W5, CA

(73) Assignee: ECOLE NATIONAL SUPERIEURE D'ARTS ET METIERS (ENSAM, Paris (FR)

(21) Appl. No.: 12/919,857

(22) PCT Filed: Feb. 29, 2008
(86) PCT No.: PCT/IB2008/051807

§ 371 Date: Aug. 27, 2010

(51) Int. Cl. A61B 5/05; G06N 5/00

(52) U.S. Cl. 600/407; 706/45; 703/2

(57) ABSTRACT

A method comprising: -providing acquisition data (34,38) of a patient, comprising acquisition data of a bone structure of the patient in a given position, providing a knowledge base (22,43,47), comprising data related to such bone structures, -determining a patient-specific position-specific finite-element model of the bone structure comprising: patient-specific three-dimensional geometry, patient-specific mechanical characteristics of the bone structure, and patient-specific position-specific mechanical load applied to the bone structure in the position.

### CROSS-REFERENCE

The present application is the U.S. National Stage of PCT/IB2008/051807 filed Feb. 29, 2008, the entirety of which is incorporated herein by reference.

### FIELD OF THE INVENTION

The instant invention relates to medical imaging methods and systems for providing a finite-element model.

### BACKGROUND OF THE INVENTION

When it comes to mechanical structures, the advantages of the finite-element-method are well known. Recently, attempts were made to use this method to simulate the behaviour of body structures, in particular bony structures of a patient as a mechanical system. Such simulations are difficult: it is necessary to obtain a precise geometry of the bony structure, the mechanical properties of the internal body organs, and the applied loads (also called “boundary conditions”). An attempt to provide such a finite-element-model is described in U.S. Pat. No. 5,172,695. In this patent, the three-dimensional geometry is obtained using a conventional Computer Tomography scanner. The volumetric image provided by the scanner is also a way to quantify the local mechanical properties, by linking the grey value of the voxel to the bone mineral density, which itself can be correlated to the mechanical characteristics of the bone. Then, a finite-element calculation is performed by applying loads on a bony surface.

There is a need to improve the clinical relevance of such calculations, so that their results could be used by clinicians.

### SUMMARY OF THE INVENTION

To this aim, according to the invention, a method is provided which comprises:
providing acquisition data of a patient, comprising at least acquisition data of a bone structure of the patient in a given functional position,
providing a knowledge base, comprising at least data related to structures of the same type as the bone structure of the patient,
determining, at least from said acquisition data, a patient-specific three-dimensional geometry,
determining, from said acquisition data and said knowledge base, patient-specific mechanical characteristics of the bone structure, and
determining, at least from the acquisition data, a patient-specific position-specific mechanical load applied to the bone structure in said given functional position,
assembling the three-dimensional geometry, the mechanical characteristics and the mechanical load into a patient-specific position-specific finite-element model of the bone structure.

With these features, it is possible to take into account the position of the patient during the acquisition, so that the applied load used for the calculation corresponds to the one actually applied on the bony structure in this functional position.

In some embodiments, one might also use one or more of the features defined in the dependent claims. Advantages of some of these embodiments include reduction of the radiation dose applied to the patient.

### BRIEF DESCRIPTION OF THE DRAWINGS

Other characteristics and advantages of the invention will readily appear from the following description of two of its embodiments, provided as a non-limitative example, and of the accompanying drawings.

On the drawings:

FIG. 1 is a perspective view of an example of a medical imager according to an embodiment of the invention,

FIG. 2 is a schematic view of the computerized system of FIG. 1,

FIG. 3 is a perspective view of an example of a generic model for use in a method according to an embodiment of the invention,

FIG. 4 is a schematic view of a computer screen on which detection data is schematically represented,

FIG. 5 is a schematic view of an FEM model,

FIGS. 6a and 6b are schematic views with a basic muscular model, illustrating how the loads can be estimated using the patient position-specificity,

FIG. 7 is a cross-sectional view through a patient, showing a plurality of applied muscular forces illustrating data related to a more sophisticated muscular model,

FIG. 8 is a schematic view of a result of a FEM calculation for a FEM model such as the one of FIG. 5, and

FIG. 9 is a schematic perspective view of another possible embodiment for an installation.

On the different figures, the same reference signs designate like or similar elements.

### DETAILED DESCRIPTION

FIG. 1 shows a radiographic apparatus 1 for three-dimensional reconstruction, the apparatus comprising a moving frame 2 displaceable under motor drive along vertical guides 3 in both directions of translation 3a.

The frame surrounds a field of observation 4 in which a patient P may be placed in a given position, e.g. standing, for observing an osteo-articular structure of the patient when in the standing position, which may be relevant for patients suffering from postural imbalance for example.

The moving frame 2 carries a first radiological source 5 and a first detector 6 which is placed facing the source 5 beyond the field 4, and which comprises at least one horizontal line 6a of detector cells. By way of example, the detector 6 may be a gas detector responsive to low doses of radiation, e.g. as described in documents FR-A-2 749 402 or FR-A-2 754 068. Naturally, other types of detectors may optionally be used in the context of the present invention.

The radiological source 5 is adapted to emit ionizing radiation, in particular X-rays, suitable for being detected by the detector 6 in an image-taking direction 7 that is antero-posterior relative to the patient P, the rays passing through a horizontal slit 8 made through an aiming mask 9 such as a metal plate in order to generate a horizontal beam 10 of ionizing radiation in the field of observation 4.

The moving frame 2 also carries a second radiological source 11 similar to the source 5 and a second detector 12 similar to the detector 6, disposed facing the source 11 beyond the field 4, and comprising at least one horizontal line 12a of detector cells.

The radiological source 11 is adapted to emit ionizing radiation in a image-taking direction 13 that is lateral relative to the patient P, passing through a horizontal slit 14 formed in an aiming mask 15 such as a metal plate in order to generate a horizontal beam 16 of ionizing radiation in the field of observation 4.

Naturally, there could be more than two radiological sources and detectors, and the image-taking directions of these various radiological sources could, where appropriate, be other than mutually perpendicular, and they need not even be horizontal.

One or each source 5, 11 can be made to emit a beam of X-rays distributed on two different energy peaks, for example by the use of different filters. For example, the source 5 emits a beam of low energy and a beam of high energy alternately during the vertical scanning. The obtained image carries both high energy and low energy acquisition.

In the present example, the patient P is standing on a force-platform 42 which enables to provide the weight and the position of the gravity line of the patient. In another embodiment, the weight data of the patient could be measured by a separate device, before the patient steps into the radiographic apparatus.

The two detectors 6, 12 are connected to a computerized system 37 or some other electronic control system provided with:
an input interface comprising at least a keyboard 18 and generally also a mouse (not shown);
an output interface comprising at least a screen 19; and
a processor 17, for executing a computer program adapted to implement the method described herein.

The computerized system 37 may also be connected to the motor-driven drive means (not shown) contained in the guide 3, and to the sources 5 and 11, so as to control the vertical displacement of the frame and/or the emission of ionizing radiation.

By way of example, the method which is described below is a method of obtention of a finite-element model of a vertebra using a knowledge base of vertebrae. However, this method could be used for any bone or osteo-articular structure of the body such as for example, the femur, the upper limb, the lower limb, the hip, or even part or totality of the skeleton, when a knowledge base of the structure to be reconstructed is provided.

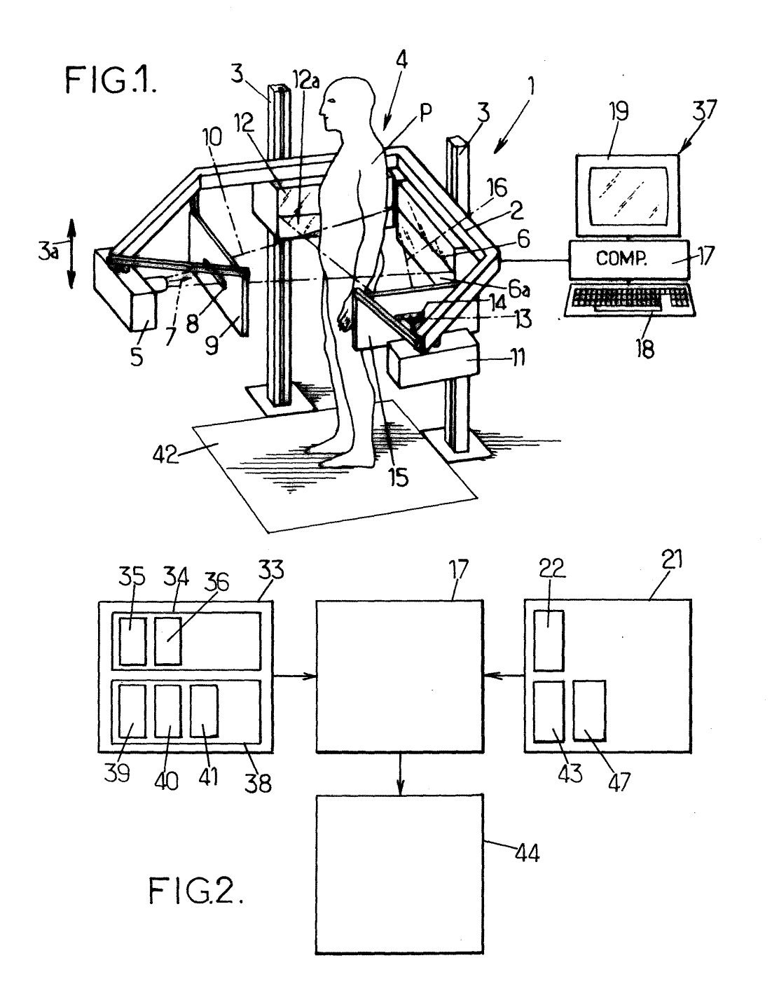

As an example shown on FIG. 2, the processor 17 is connected to a detection memory 33 comprising detection data of the patient P.

The detection data of the patient P comprises detection data 34 of the bone structure of the patient, such as, for example, antero-posterior 35 and lateral 36 (possibly dual-energy) X-ray images of the patient taken with the apparatus 1. In some embodiments, the detection data can further comprise external detection data 38 of the patient such as, for example, antero-posterior 39 and lateral 40 detection of the external envelope (skin) of the patient detected on the antero-posterior and lateral radiographs, respectively, and/or, for example, data of the 3D envelope of the patient and/or weight data 41 of the patient, for example provided from the force platform 42.

The processor 17 is further connected to a knowledge base memory 21 comprising a knowledge base 22 of the geometry of the bone structure, a knowledge base 43 related to the mass and the location of the center of gravity of patient body segments with regard to the bone(s) of interest, and a knowledge base 47 of bone mechanical characteristics. There are multiple ways in which the knowledge can be stored in the knowledge base(s).

As will be described in more details below, the processor 17 calculates, from the detection data and the knowledge base a patient-specific position-specific finite-element model (FEM) which can be used in an appropriate FEM-solver 44. Such FEM-solvers are commercially available and will not be described in more details.

The detection memory 33, the processor 17, the knowledge base memory 21 and the FEM-solver 44 could be found on the same computerized system, or distributed over a network.

For the bone structure under study, the knowledge database may be constructed as or from data obtained from similar structures.

The geometry knowledge database 22 is arranged so as to store surface data relating to coordinates of points belonging to a surface of a generic model.

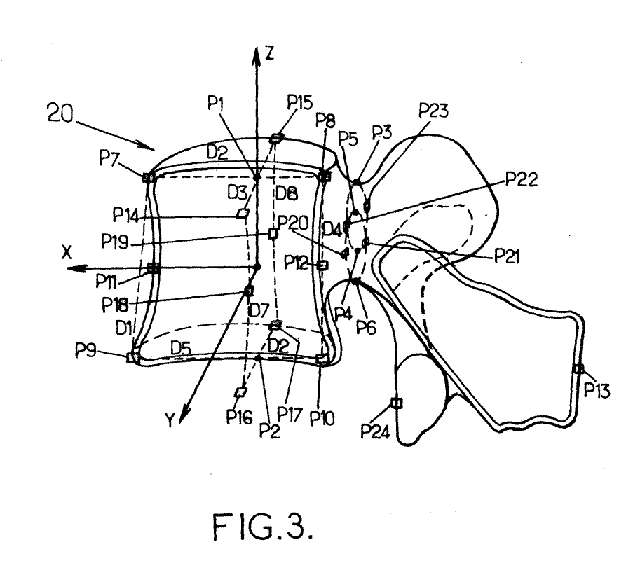

In this example, the surface data comprise information about points corresponding to particular reference marks on structures of the same type of the studied structure, acquired beforehand, for example by computer tomography. By way of example, the geometry knowledge database 22 of the vertebra contains the coordinates x_{P1}, y_{P1}, z_{P1}, . . , x_{P23}, y_{P23}, z_{P23}, of characteristic points P1, . . , P23 for the vertebra of each of a plurality of previous patients, characteristic lengths D1 . . . D8 for each vertebra, as shown in FIG. 3, segments, straight lines or arcs that are characteristics of the object, and/or outlines and edges of these particular vertebra.

As shown in FIG. 3, the coordinates of characteristic points or lines may be expressed, for example, in a local X, Y, Z frame of reference.

Further, the outer and inner surface data may further comprise a mesh of several hundred to several hundred thousand points and/or elements of an average vertebra.

It is possible to establish a subset (not represented) of the knowledge database related to vertebrae belonging to healthy individuals or to individuals suffering from different pathologies, and similarly it is possible to characterize each vertebral mesh as a function of the weight, the size, the age, or any other type of parameter concerning the individual that is deemed to be necessary.

Alternatively, in a not represented embodiment, the vertebral mesh may be obtained from a mathematical model constructed from the previously acquired data. By way of example, the surface data may include statistical data (means, variances . . . ) for each parameter of the knowledge base. The surface data may include mathematical equations for determining from the knowledge base of a given vertebra, the positions of the characteristic points for a personalised mesh on the basis of values of estimator parameters for said object. For example, the coordinates of the characteristic points may be parameterized by functions of these parameters.

As used herein, the term “data” represents as well raw data such as obtained from measurements of similar structures, and elaborated data such as equations or the like obtained from the observation of such measurements. The word “data” is used to represent any knowledge which can be stored on a medium and relate to the studied structure.

The mechanical characteristic knowledge data base 47 can for example have been previously obtained from imaging samples from anatomical specimen by X-rays, so as to determine the grey-level of the sample (the bone's mineral density), then by determining in vitro the bone's mechanical properties by mechanical testing (compression, traction and/or bending tests), so as to correlate the grey level and the mechanical characteristics of the sample.

Referring back to FIG. 1, the computer 37 is used initially to take two radiographic images of the patient P by causing the field of observation 4 to be scanned by the beams 10 and 16 of ionizing radiation over a height corresponding to the structure of the patient that is to be observed, for example the spine and the pelvis, or indeed the entire skeleton. For this purpose, the frame is displaceable for example over the height of the patient.

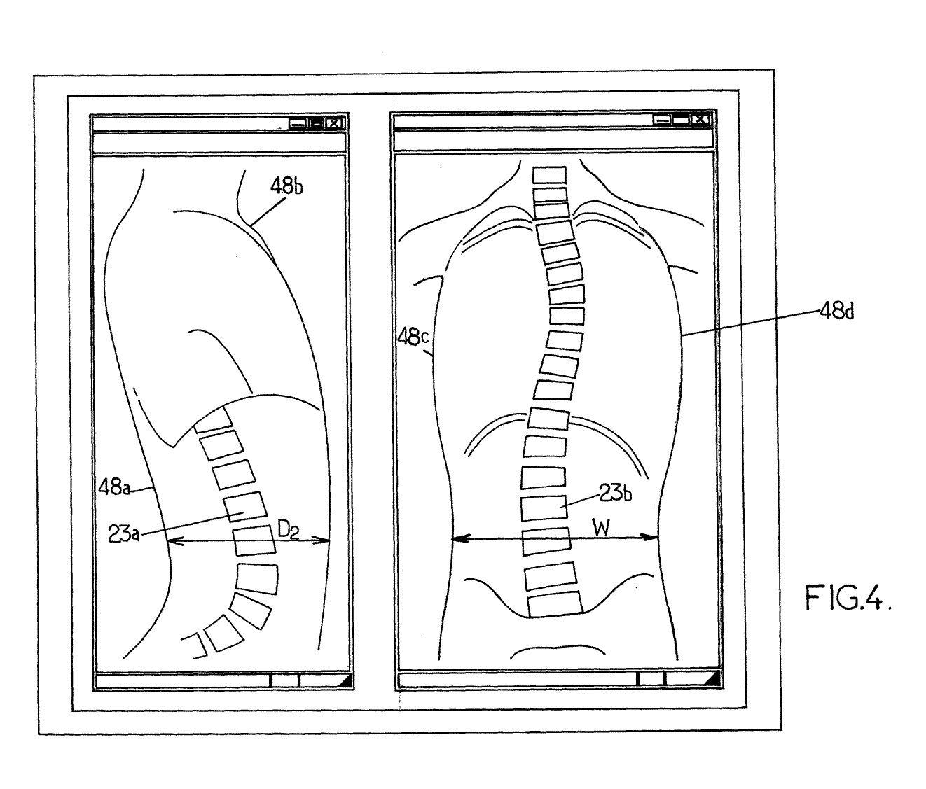

During this movement, two calibrated digital radio-graphic images of the portion of the patient under examination are stored in the memory 33 of the microcomputer 37, for example an antero-posterior image and a lateral image respectively, wherein each images can be viewed on the screen 19 of the microcomputer, as shown schematically in FIG. 4. Of course, FIG. 4 is just an illustrative schematic representation of radiographs, which are grey-levelled images.

The processor 17 receives two-dimensional patient-specific detection data 35, 36, i.e. the two radiographic images of the patient P. The processor 17 comprises or communicates with the memory 21 that stores the knowledge database of the structure 22 shown in FIG. 2.

The processor selects an initial model in the geometry knowledge database 22 of the bone from the detection data 35, 36. The selection step may take into account parameters such as a previous radiography of a same patient, a knowledge of the part of the patient that is being imaged etc. to identify an initial model.

In an alternative embodiment, the selection step is performed by an operator, who selects an initial model, and might also point out parts 23a, 23b of the images corresponding to the model.

In an alternative embodiment, the method is to be used for imaging a single type of structure, e.g. femurs, and the base 22 may contain data relative to a single model, e.g. of an healthy femur. In this case, the selection step is trivial.

The invention is by no means limited by the implementation of the selection step.

The 3D outer surfacic model could be obtained by already known methods such as the one described in Laporte et al. “A Biplanar Reconstruction Method Based on 2D and 3D Contours: Application to the Distal Femur”, Computer Methods in Biomechanics and Biomedical Engineering 6(1), 1-6, 2003, which is hereby incorporated by reference in its entirety for all purposes.

Although this article is related to the distal femur, the method described there could be used for any other bone structure. This exemplary method could be summarized as follows:
the initial model is placed in virtual space so as to roughly correspond to the images of the structure,
anatomical regions are defined on the generic model,
2D contours Λⱼ are identified on the radiographs,
2D contours Ωⱼ in the plane of the radiographs are generated from the initial model,
contours Ωⱼ and Λⱼ are associated to one another,
the initial model is deformed (for example by combination of a rigid transformation and an homothetic transformation) and contours Ωⱼ are calculated for the deformed model, until contours Ωⱼ and Λⱼ fit,
the deformed model is further deformed so as to be optimized, for example by kriging.

A three-dimensional position-specific patient-specific surfacic geometric model of the bone structure of the patient is thereby obtained in the frame of reference of the imager. This model is represented as a mesh comprising, for example, surfacic shell elements 46, for representing the cortical bone. For example, the inside volume can be meshed with internal (not shown) volumic elements, for representing the trabecular bone. In addition, the model is obtained in the frame of reference of the imager, i.e. corresponds to the actual position of the patient when taking the image.

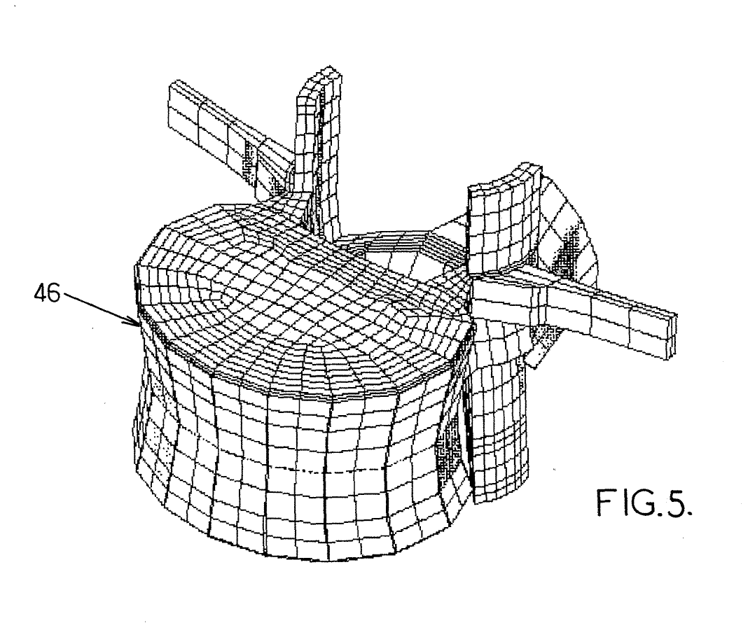

On FIG. 5, the patient specific resulting mesh is represented very schematically.

In a further step, patient-specific bone mechanical properties (e.g. Young's modulus, maximum compressive or tensile strength) are input into the model. Further parameters, such as those related to the anisotropy of the bone material could also be defined. These parameters could be defined individually for each element of the mesh or for parts of the model having, in average, the same mechanical behaviour.

The mechanical property knowledge database 47 is used to correlate the information provided from the detection data, such as the grey level in the radiographic images, to the mechanical characteristics of the bone.

For example, for each surfacic element of the obtained geometric model, a thickness value and a value of the Young's modulus are defined from the mechanical characteristics knowledge base 47. For example, initially, uniform thickness and Young's modulus data are defined over the model elements, based on average thickness and Young's modulus data provided from the mechanical characteristics knowledge base 47.

In variant embodiments, different initial values could be defined for different anatomical regions such as the anterior, lateral, and posterior vertebral walls, the pedicle, etc. . . .

It should be noted that other mechanical parameters could be defined, such as a single parameter combining thickness and Young's modulus (equivalent homogenized modulus for a given thickness).

The initial mechanical characteristics inserted into the geometric model can be modified to be made patient-specific. For example, if we take into account the fact that the grey level on the image is related both to attenuation coefficient of the tissues (which is also related density and thus to elastic modulus) and cortical thickness. We can simulate virtual X-rays according to the initial attenuation/thickness and we can change those initial parameters until the simulated radiographs are made as similar as possible to the real radiographs. This step will lead to patient-specific mechanical properties.

The above is just an example of how to determine the patient's specific geometry and mechanical characteristics. The mechanical characteristics could be determined simultaneously with the determination of the geometry, by using the mechanical characteristics already during the reconstruction step.

In another embodiment, the cortical bone could also be meshed with volumic elements.

Further, the patient-specific position-specific mechanical loads applied to the bone structure in the position of the patient when taking the acquisition data are estimated. For example, these loads are due to gravity. The patient-specific mechanical loads can be assessed from any suitable kind of mechanical model.

For example, the body is virtually divided into segments, such as the head, each upper limb, the trunk, and each of the lower limbs. The trunk segment could be divided in sub-segments, for example at each vertebral level and at the pelvis level. For each segment or sub-segment, the mass and position of the centre of gravity is determined from the geometry at that level. For example, the knowledge base 43 related to the mass and location of the centre of gravity of the body segments comprise an anthropometric table enabling to estimate, from the local geometry, the mass and the location of the centre of gravity of the studied segment or sub-segment. The local geometry could for example be the bone geometry obtained from the radiographs or from the previously obtained mesh, for example.

In this case, the location of the centre of gravity and the mass of the sub-segment can be statistically determined from the knowledge base 43 based only on the location of the bone in one, or both radiographs or on the 3D mesh.

In another embodiment, the location of the centre of gravity and the mass of the sub-segment can be statistically determined using additional data 39, 40 relating to the external envelope of the patient at the sub-segment.

For example, the knowledge base 43 comprises knowledge enabling to determine, for a given envelope, the location of the centre of gravity and the mass of each corporal sub-segment situated above the bony structure of interest.

In this example, as shown on FIG. 4, parts of the patient's envelope are determined. For example, the back and front outlines 48a, 48b of the skin of the patient are detected on the lateral radiographs, and left and right outlines 48c, 48d on the frontal radiograph. These outlines could be determined, either automatically by image processing, or manually by the user. For example, for each sub-segment, the lateral depth between the front and back envelope is measured as shown by arrow D_{L} on FIG. 4, and the width between the left and right envelopes is measured, as shown (for another sub-segment) by arrow W on FIG. 4. The knowledge base 43 comprises a priori knowledge of the average density of the sub-segment which allows calculating the mass and the position of the centre of gravity of this sub-segment, as a function of depth and width. This a priori knowledge is used, together with the patient specific measurements, to determine the patient-specific position of the sub-segment's centre of gravity and its mass.

This average density might not distinguish density values for bone and soft tissue and, in this case, is an overall density for the sub-segment. In a variant embodiment where bone detection data is available for the sub-segment, the stored density might distinguish between average soft tissue density and average bone density.

Although the above embodiments describe using both the width and depth of the sub-segment for determining its mass and the location of its centre of gravity, only one of the width and depth could be used, in a variant embodiment for this determination.

This determination is performed for all the sub-segments and segments above the studied structure, so as to evaluate the location and intensity of the gravitational load on the studied structure, by calculating the barycentre of all the segments above the structure.

There are many other possible embodiments for obtaining the mass and the location of the centre of gravity. For example, the medical imager could comprise an optical acquisition device, such as one or more cameras, the images of which can be determined in the same three-dimensional frame of reference as the one of the geometrical 3D model, of the studied structure so as to reconstruct a full 3D envelope. An embodiment could use the projection of structured light onto the patient, detection of this light by cameras, and the processing of the detected optical images by suitable well-known algorithms.

The envelope data is used to determine, in the imaging position (here: standing), the point of application of the gravitational load of the part of the patient situated above the bone structure of which a 3-dimensional reconstruction has been previously established.

In any of the above embodiments, the total mass 41 of the patient could also be used for the determination of the mass and the location of the centre of gravity of the sub-segment.

In a variant embodiment, the mass and the location of the centre of gravity of the segments under the studied structure could be determined using any of the above methods. Then, the mass and the location of the centre of gravity of the part above the studied structure could be determined therefrom and from the data provided from the force platform 42, by an equilibrium calculation.

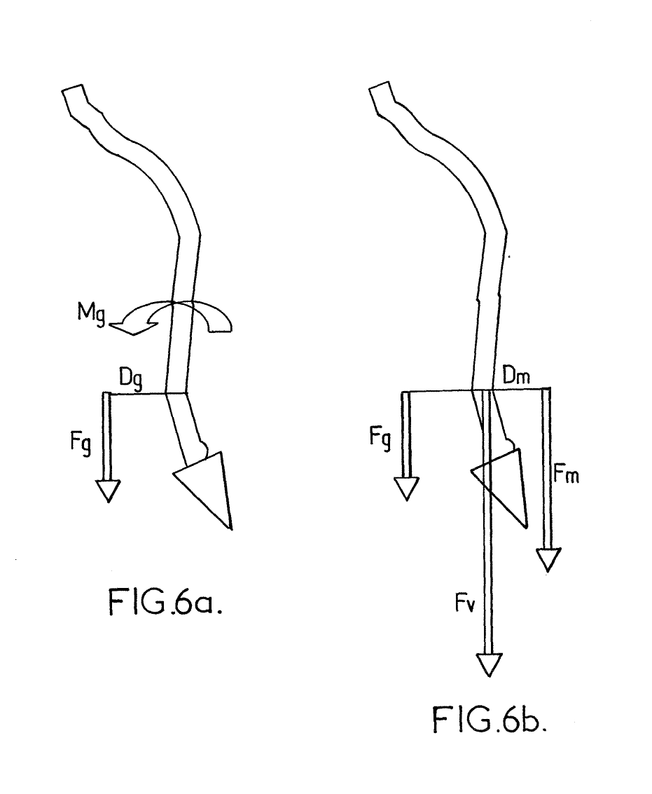

The applied load F_{g} on the bone structure is calculated by the total mass above the bone structure, applied on the centre of mass G of the portions of the patient above the bone structure, and along the vertical direction. It is thus shown on FIG. 6a that the gravity load for a given vertebra is applied at a distance Dg from the vertebra, whereby a moment Mg is applied on the vertebra. As shown on FIG. 6b, this moment can be regulated by a muscular force Fₘ, which is applied at a distance Dm from the vertebra. F_{g} and Fₘ build up the total resultant force Fᵥ applied on the vertebra.

There is a whole range of possibilities to determine the muscular load. One might use a simple muscular model in which only one set of muscles is activated.

More complex models can be used. Taking into account the muscles lever arms (the distance between the gravity line and the bone and the distance between muscle attachments and the bone) it is possible to estimate the loads on the bone. Such mechanical model could be improved using a multiple muscles regulation model. For example a proprioception based regulation model can be proposed to estimate the trunk muscular loads. The muscular model is built as a closed loop regulation system considering the load components within the intervertebral joint as the commands that have to be maintained beyond given thresholds, such as described for example in “A proprioception based regulation model to estimate the trunk muscular forces”, Pomero and al., Computer Methods in Biomechanics and Biomedical Engineering, Vol. 7, No 6, December 2004, pages 331-338, which is hereby incorporated by reference in its entirety for all purposes.

The initial mesh of the generic model could bear information of the nodes or elements which are to be considered as muscle insertion points into the bone. Upon deforming the initial mesh to be rendered patient-specific, these nodes and elements are displaced and/or deformed, which provides with a patient-specific location of muscle insertion.

As shown on FIG. 7, arrows 51 represent the intensity of forces exerted by the muscles 52 in the sectional view, as determined from the muscular model. The resulting force 53 and torque 54 at the bone joint are also shown on FIG. 7. FIG. 7 is a purely illustrative view of the muscular loads determined by the muscular model, but is not a view obtained from the medical imager of FIG. 1.

The thereby determined loads are expressed in the same frame of reference as the 3D patient-specific mesh with personalised mechanical characteristics.

Finally, the applied loads are input into the finite-element model as shown by arrow Fᵥ on FIG. 6b. The above are just examples of possible patient-specific loads and only examples of how to measure them.

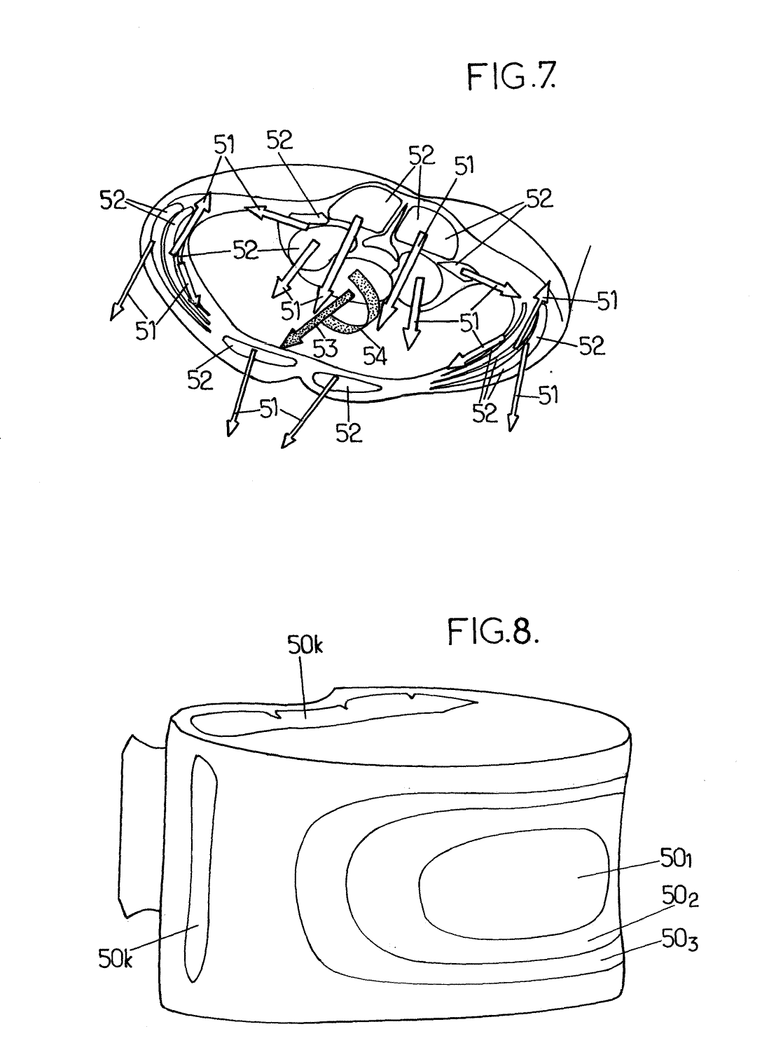

The thus obtained patient-specific and position-specific finite-element model is input in a finite element solver 44, which solves the mechanical problem for the model. The results can be displayed on the screen 19, for example in pseudo-colours, such as schematically illustrated on FIG. 8, on which a level of stress experienced by the patient in the standing position in which he was imaged is represented. The highest stress is found in region 50₁. Decreasing stresses are encountered in regions 50₂, 50₃, . . . , 50ₖ, . . . These results could allow a clinician to visualize on which part of the bone the applied mechanical load has a critical effect in the functional position in which the patient was imaged. Automatic post-processing could be made to determine biomechanically relevant parameters, such as, for example, the highest stress or highest strain encountered in an element, an average stress in a given part of the bone model, a number of contiguous elements having a stress or strain above a predetermined threshold, or any pre-established biomechanical criteria.

These biomechanical parameters could be taken into account by the clinician to determine a risk of fracture for the patient, for example osteoporotic patients and/or to propose a therapy.

The method which was described above was by way of example only. The structure to be studied could be a single bone or a whole osteo-articular structure and could be any part of the body skeleton of a patient, either in a lying or standing position or any other functional position in which gravity induces a critical load on the bone structure (squatting, standing carrying a load, . . . ).

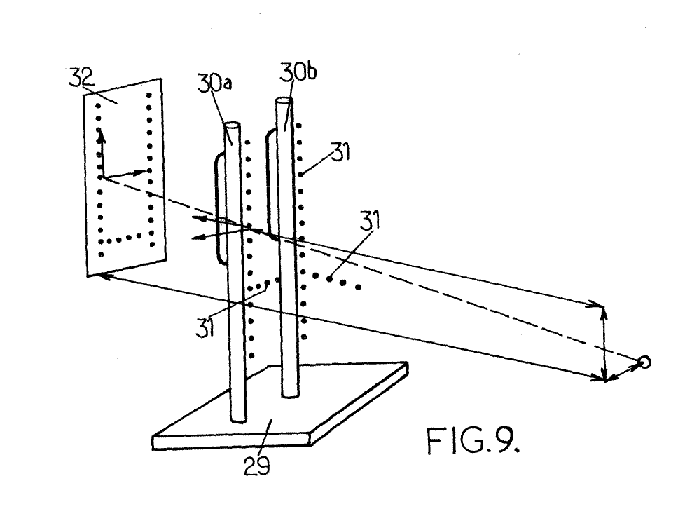

In particular, the acquisition scheme is not limited to the one presented in relation to FIG. 1, wherein a lateral and an antero-posterior images of the structure are obtained simultaneously. One could use other kind of acquisition apparatus, such as the one shown on FIG. 9 comprising a platen 29 and two posts 30a, 30b extending vertically and comprising radio-opaque markers 31 positioned in three-dimensional space so that their detection on the obtained detection data 32 will be used for calibrating the images respective to each other. The platen 29 will be movable relative to the radiological source in order to take a plurality of images of the patient standing on the platen along different orientations. It is even estimated that the present method could be performed using, as detection data, either a pair of non-calibrated radiographs, or a single radiograph.

1. A method comprising:
providing acquisition data of a patient, comprising at least acquisition data of a bone structure of the patient in a given functional position,
providing a knowledge base, comprising at least data related to structures of the same type as the bone structure of the patient,
determining, at least from said acquisition data, a patient-specific three-dimensional geometry of the bone structure,
determining, from said acquisition data and said knowledge base, patient-specific mechanical characteristics of the bone structure,
determining, at least from the acquisition data, a patient-specific position-specific mechanical load applied to the bone structure in said functional given position,
assembling the three-dimensional geometry, the mechanical characteristics and the mechanical load into a patient-specific position-specific finite-element model of the bone structure.

2. Method according to claim 1 wherein the knowledge database comprises at least data related to the three-dimensional geometry of structures of the same type as the structure of the patient, and wherein the patient-specific three-dimensional geometry is determined using the knowledge base.

3. Method according to claim 1 wherein the knowledge database comprises at least data related to the mechanical characteristics of structures of the same type as the structure of the patient.

4. Method according to claim 1 wherein the knowledge database comprises at least data related to mass and/or location of a centre of mass of body segments, and wherein the patient-specific position-specific mechanical load is determined using the knowledge base.

5. A method according to claim 4, wherein acquisition data further comprise acquisition data of at least a part of the external envelope of the patient,
wherein the knowledge base further comprise data related to mass and/or location of a centre of mass correlated to external envelopes.

6. A method according to claim 1, wherein providing acquisition data further comprises providing the weight of the patient.

7. A method according to claim 1, wherein the mechanical load comprises the load of the mass of the patient situated above the bone structure in said functional position.

8. A method according to claim 7 wherein the mechanical load further comprises a muscular load applied to the bone structure.

9. A method according to claim 1 wherein acquisition data of the bone structure comprises at least one bi-dimensional image of the bone structure, and wherein the knowledge base comprises at least data related to the mechanical characteristics of bone structures of the same type as the bone structure of the patient.

10. A method according to claim 1 wherein acquisition data of the bone structure comprises at least one bi-dimensional image of the bone structure, and wherein the patient-specific three-dimensional geometry is obtained by fitting a generic three-dimensional mesh of the bone structure to the image, said 3D mesh comprising at least one 3D surface.

11. A method according to claim 9, wherein acquisition data of the bone structure comprises at least two bi-dimensional images of the bone structure taken along two non-parallel directions, and referenced in the same reference frame.

12. A method according to claims 9, wherein the images are X-ray images.

13. A method according to claim 1, further comprising a step of placing the patient in the given functional position in a medical imager, and of acquiring at least said acquisition data of the bony structure.

14. A method according to claim 13 further comprising a step of detecting a part of the external envelope of the patient in said given position.

15. A method according to claim 1, further comprising applying a finite-element-method solver to the patient-specific position-specific model.

16. A method according to claim 15, further comprising determining with the finite-element-method solver a biomechanical criteria of the bone structure.

17. Computer program product comprising instructions for causing a programmable unit to perform the method of claim 1 when executed on said programmable unit.

18. A system comprising:
a medical imager adapted to provide acquisition data of a patient, comprising at least acquisition data of a bone structure of the patient in a given position,
a knowledge base, comprising at least data related to structures of the same type as the structure of the patient,
a computerized unit adapted to determine, from said acquisition data a patient-specific three-dimensional geometry of the bone structure,
wherein the computerized unit is further adapted to determine from the acquisition data and said knowledge base, a patient-specific mechanical characteristics of the bone structure,
wherein the computerized unit is further adapted to determine, at least from the acquisition data a patient-specific position-specific mechanical load applied to the bone structure in said given functional position,
wherein the computerized unit is further adapted to assemble the three-dimensional geometry, the mechanical characteristics and the mechanical load into a patient-specific position-specific finite-element model of the bone structure.

19. A system according to claim 18 further comprising a finite-element-method solver adapted to be applied to the patient-specific position-specific finite-element model.

20. A system according to claim 18 wherein the imager comprises at least a first X-ray source, a first X-ray detector, and a patient-receiving space adapted to receive the patient in standing position between the first source and the first detector, said detector being adapted to detect said acquisition data of the bone structure of the patient.

21. A system according to claim 20, wherein the first X-ray source and the first X-ray detector are aligned along a first direction, wherein the imager further comprises a second X-ray source and a second X-ray detector aligned along a second direction not parallel with the first direction.

22. A system according to claim 20, further comprising a displacement device adapted for moving said sources and detectors along a third direction in order to scan the patient.

23. A system according to claim 18, wherein the medical imager further comprises a force platform adapted to detect the gravity axis passing through the patient.

24. A system according to claim 18, wherein the medical imager further comprises at least one camera for detecting at least part of the external envelope of the patient.
